(19) United States
(12) Patent Application Publication
(10) Pub. No.: US 2009/0162245 A1
(43) Pub. Date: Jun. 25, 2009

(54) HANDHELD GAMING MACHINE SANITITZING SYSTEM

(75) Inventor: James M. Rasmussen, Chicago, IL (US)

Correspondence Address: NIXON PEABODY LLP, 161 N CLARK ST., 48TH FLOOR, CHICAGO, IL, 60601-3213, US

(73) Assignee: WMS GAMING, INC., Waukegan, IL (US)

(21) Appl. No.: 12/305,919

(22) PCT Filed: Jun. 8, 2007
(86) PCT No.: PCT/US07/13566

§ 371 Date: Dec. 19, 2008

Related U.S. Application Data
(60) Provisional application No. 60/815,201, filed on Jun. 20, 2006.

(51) Int. Cl. A61L 2/10; A61L 2/00; B01J 19/12; A61L 2/20

(52) U.S. Cl. 422/24; 422/300; 422/186.3; 422/28

(57) ABSTRACT

A sanitizing system for a handheld gaming device is provided. The sanitizing system comprising a housing, a door, a gas supply, and a control system. The housing defines at least a chamber, a gas inlet aperture, and an opening for receiving the handheld gaming device. The door is configured to seal the opening. The gas supply comprises a sanitizing gas and is connected to the gas inlet aperture. The control system is configured to selectively introduce gas from the gas supply into the chamber.

### FIELD OF THE INVENTION

The present invention relates generally to handheld wagering games and handheld machines for playing wagering games, and more specifically to an apparatus for sanitizing handheld wagering games and methods thereof.

### BACKGROUND OF THE INVENTION

Gaming machines, such as slot machines, video poker machines and the like, have been a cornerstone of the gaming industry for several years. Generally, the popularity of such machines with players is dependent on the likelihood (or perceived likelihood) of winning money at the machine and the intrinsic entertainment value of the machine relative to other available gaming options. Where the available gaming options include a number of competing machines and the expectation of winning at each machine is roughly the same (or believed to be the same), players are likely to be attracted to the most entertaining and exciting machines.

Shrewd operators consequently strive to employ the most entertaining and exciting machines, features, and enhancements available because such machines attract frequent play and hence increase profitability to the operator. Therefore, there is a continuing need for gaming machine manufacturers to continuously develop new games and improved gaming enhancements that will attract frequent play through enhanced entertainment value to the player.

In recent years, handheld electronic devices (e.g., telephones, personal data assistances (PDAs), handheld computers, handheld gaming machines, etc.) have become ubiquitous. These handheld devices advantageously permit the user to interact with the device at a time and place of the user's choosing, rather than requiring the user to be immobile at a fixed point (e.g., a fixed telephone, a desktop computer, a television-based or CRT monitor-based gaming device). The playability and appeal of conventional wagering games is improved by rendering such wagering games mobile through an appropriate portable electronic wagering game interface (i.e., a handheld gaming device).

However, handling and environmental exposure potentially renders these devices in an unacceptable state of cleanliness. Thus, there exists a need to provide a safe and aesthetically pleasing handheld gaming device for subsequent use.

### SUMMARY OF THE INVENTION

A sanitizing system for a handheld gaming device is provided, according to one aspect of the present invention. The sanitizing system comprises a housing, a door, a gas supply, and a control system. The housing defines at least a chamber, a gas inlet aperture, and an opening for receiving the handheld gaming device. The door is configured to seal the opening. The gas supply comprises a sanitizing gas and is connected to the gas inlet aperture. The control system is configured to selectively introduce gas from the gas supply into the chamber.

According to another aspect of the present invention, a method for sanitizing a handheld gaming device is also provided. The method comprises the acts of moving the handheld gaming device into a chamber, substantially sealing the chamber, and sanitizing the handheld gaming device.

According to another aspect of the present invention, a sanitizing system for a handheld gaming device is provided. The sanitizing system includes a housing that defines a chamber and an opening for receiving a handheld gaming device. The sanitizing system further includes a partition configured to occlude the opening. The sanitizing system additionally includes at least one ultraviolet lamp disposed either adjacent to or within the chamber to introduce ultraviolet light into the chamber to at least partially sanitize the handheld gaming device disposed therein.

Additional aspects of the invention will be apparent to those of ordinary skill in the art in view of the detailed description of various embodiments, which is made with reference to the drawings, a brief description of which is provided below.

### BRIEF DESCRIPTION OF THE DRAWINGS

FIG. 1a is a perspective view of a free standing gaming machine embodying the present invention.

FIG. 1b is a perspective view of a handheld gaming machine embodying the present invention.

FIG. 2 is a block diagram of a control system suitable for operating the gaming machines of FIGS. 1a and 1b.

FIG. 3 is a schematic illustration of a sanitizing system.

FIG. 4 is a schematic illustration of the sanitizing system of FIG. 3 including a handheld gaming machine, before use.

FIG. 5 is a schematic illustration of the sanitizing system of FIG. 3 including a handheld gaming machine, in use.

FIG. 6 is a schematic illustration of a sanitizing system, according to an alternative embodiment.

### DETAILED DESCRIPTION

While this invention is susceptible of embodiment in many different forms, there is shown in the drawings and will herein be described in detail preferred embodiments of the invention with the understanding that the present disclosure is to be considered as an exemplification of the principles of the invention and is not intended to limit the broad aspect of the invention to the embodiments illustrated.

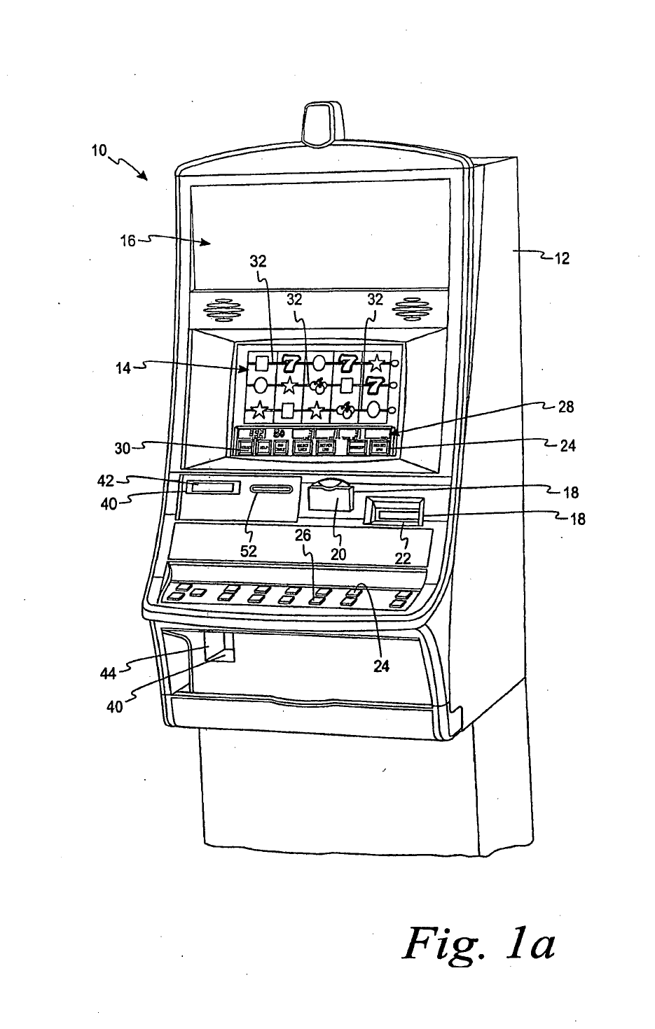

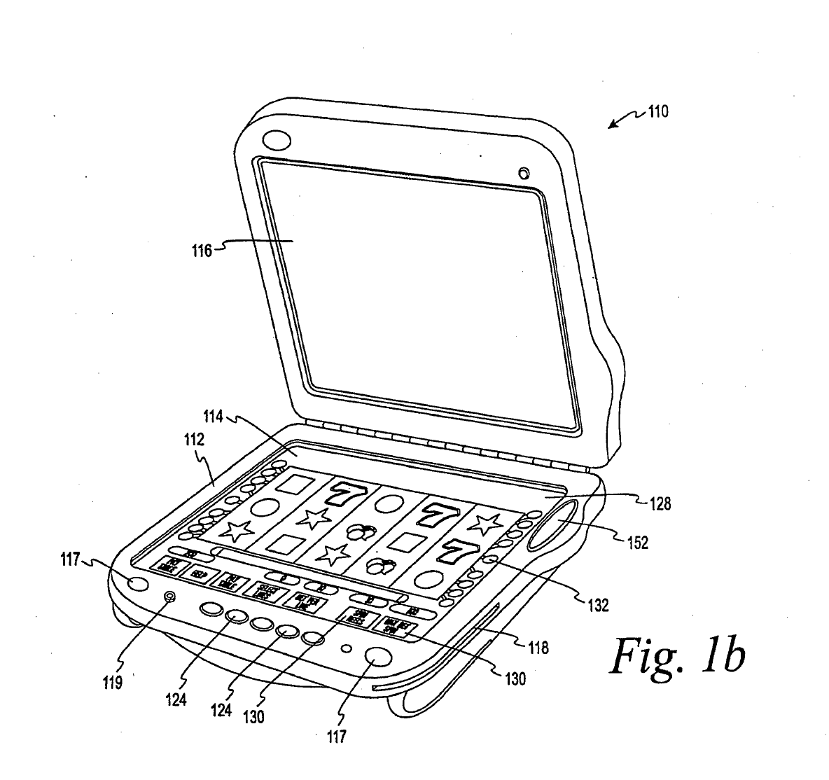

Referring to FIG. 1a, a gaming machine 10 is used in gaming establishments such as casinos. With regard to the present invention, the gaming machine 10 may be any type of gaming machine and may have varying structures and methods of operation. For example, the gaming machine 10 may be an electromechanical gaming machine configured to play mechanical slots, or it may be an electronic gaming machine configured to play a video casino game, such as blackjack, slots, keno, poker, blackjack, roulette, etc.

The gaming machine 10 comprises a housing 12 and includes input devices, including a value input device 18 and a player input device 24. For output the gaming machine 10 includes a primary display 14 for displaying information about the basic wagering game. The primary display 14 can also display information about a bonus wagering game and a progressive wagering game. The gaming machine 10 may also include a secondary display 16 for displaying game events, game outcomes, and/or signage information. While these typical components found in the gaming machine 10 are described below, it should be understood that numerous other elements may exist and may be used in any number of combinations to create various forms of a gaming machine 10.

The value input device 18 may be provided in many forms, individually or in combination, and is preferably located on the front of the housing 12. The value input device 18 receives currency and/or credits that are inserted by a player. The value input device 18 may include a coin acceptor 20 for receiving coin currency (see FIG. 1a). Alternatively, or in addition, the value input device 18 may include a bill acceptor 22 for receiving paper currency. Furthermore, the value input device 18 may include a ticket reader, or barcode scanner, for reading information stored on a credit ticket, a card, or other tangible portable credit storage device. The credit ticket or card may also authorize access to a central account, which can transfer money to the gaming machine 10.

The player input device 24 comprises a plurality of push buttons 26 on a button panel for operating the gaming machine 10. In addition, or alternatively, the player input device 24 may comprise a touch screen 28 mounted by adhesive, tape, or the like over the primary display 14 and/or secondary display 16. The touch screen 28 contains soft touch keys 30 denoted by graphics on the underlying primary display 14 and used to operate the gaming machine 10. The touch screen 28 provides players with an alternative method of input. A player enables a desired function either by touching the touch screen 28 at an appropriate touch key 30 or by pressing an appropriate push button 26 on the button panel. The touch keys 30 may be used to implement the same functions as push buttons 26. Alternatively, the push buttons 26 may provide inputs for one aspect of the operating the game, while the touch keys 30 may allow for input needed for another aspect of the game.

The various components of the gaming machine 10 may be connected directly to, or contained within, the housing 12, as seen in FIG. 1a, or may be located outboard of the housing 12 and connected to the housing 12 via a variety of different wired or wireless connection methods. Thus, the gaming machine 10 comprises these components whether housed in the housing 12, or outboard of the housing 12 and connected remotely.

The operation of the basic wagering game is displayed to the player on the primary display 14. The primary display 14 can also display the bonus game associated with the basic wagering game. The primary display 14 may take the form of a cathode ray tube (CRT), a high resolution LCD, a plasma display, an LED, or any other type of display suitable for use in the gaming machine 10. As shown, the primary display 14 includes the touch screen 28 overlaying the entire display (or a portion thereof) to allow players to make game-related selections. Alternatively, the primary display 14 of the gaming machine 10 may include a number of mechanical reels to display the outcome in visual association with at least one payline 32. In the illustrated embodiment, the gaming machine 10 is an “upright” version in which the primary display 14 is oriented vertically relative to the player. Alternatively, the gaming machine may be a “slant-top” version in which the primary display 14 is slanted at about a thirty-degree angle toward the player of the gaming machine 10.

A player begins play of the basic wagering game by making a wager via the value input device 18 of the gaming machine 10. A player can select play by using the player input device 24, via the buttons 26 or the touch screen keys 30. The basic game consists of a plurality of symbols arranged in an array, and includes at least one payline 32 that indicates one or more outcomes of the basic game. Such outcomes are randomly selected in response to the wagering input by the player. At least one of the plurality of randomly-selected outcomes may be a start-bonus outcome, which can include any variations of symbols or symbol combinations triggering a bonus game.

In some embodiments, the gaming machine 10 may also include a player information reader 52 that allows for identification of a player by reading a card with information indicating his or her true identity. The player information reader 52 is shown in FIG. 1a as a card reader, but may take on many forms including a ticket reader, bar code scanner, RFID transceiver or computer readable storage medium interface. Currently, identification is generally used by casinos for rewarding certain players with complimentary services or special offers. For example, a player may be enrolled in the gaming establishment's loyalty club and may be awarded certain complimentary services as that player collects points in his or her player-tracking account. The player inserts his or her card into the player information reader 52, which allows the casino's computers to register that player's wagering at the gaming machine 10. The gaming machine 10 may use the secondary display 16 or other dedicated player-tracking display for providing the player with information about his or her account or other player-specific information. Also, in some embodiments, the information reader 52 may be used to restore game assets that the player achieved and saved during a previous game session.

Depicted in FIG. 1b is a handheld or mobile gaming machine 110. Like the free standing gaming machine 10, the handheld gaming machine 110 is preferably an electronic gaming machine configured to play a video casino game such as, but not limited to, blackjack, slots, keno, poker, blackjack, and roulette. The handheld gaming machine 110 comprises a housing or casing 112 and includes input devices, including a value input device 118 and a player input device 124. For output the handheld gaming machine 110 includes, but is not limited to, a primary display 114, a secondary display 116, one or more speakers 117, one or more player-accessible ports 119 (e.g., an audio output jack for headphones, a video headset jack, etc.), and other conventional I/O devices and ports, which may or may not be player-accessible. In the embodiment depicted in FIG. 1b, the handheld gaming machine 110 comprises a secondary display 116 that is rotatable relative to the primary display 114. The optional secondary display 116 may be fixed, movable, and/or detachable/attachable relative to the primary display 114. Either the primary display 114 and/or secondary display 116 may be configured to display any aspect of a non-wagering game, wagering game, secondary games, bonus games, progressive wagering games, group games, shared-experience games or events, game events, game outcomes, scrolling information, text messaging, emails, alerts or announcements, broadcast information, subscription information, and handheld gaming machine status.

The player-accessible value input device 118 may comprise, for example, a slot located on the front, side, or top of the casing 112 configured to receive credit from a stored-value card (e.g., casino card, smart card, debit card, credit card, etc.) inserted by a player. In another aspect, the player-accessible value input device 118 may comprise a sensor (e.g., an RF sensor) configured to sense a signal (e.g., an RF signal) output by a transmitter (e.g., an RF transmitter) carried by a player. The player-accessible value input device 118 may also or alternatively include a ticket reader, or barcode scanner, for reading information stored on a credit ticket, a card, or other tangible portable credit or funds storage device. The credit ticket or card may also authorize access to a central account, which can transfer money to the handheld gaming machine 110.

Still other player-accessible value input devices 118 may require the use of touch keys 130 on the touch-screen display 128 (e.g., primary display 114 and/or secondary display 116) or player input devices 124. Upon entry of player identification information and, preferably, secondary authorization information (e.g., a password, PIN number, stored value card number, predefined key sequences, etc.), the player may be permitted to access a player's account. As one potential optional security feature, the handheld gaming machine 110 may be configured to permit a player to only access an account the player has specifically set up for the handheld gaming machine 110. Other conventional security features may also be utilized to, for example, prevent unauthorized access to a player's account, to minimize an impact of any unauthorized access to a player's account, or to prevent unauthorized access to any personal information or funds temporarily stored on the handheld gaming machine 110.

The player-accessible value input device 118 may itself comprise or utilize a biometric player information reader which permits the player to access available funds on a player's account, either alone or in combination with another of the aforementioned player-accessible value input devices 118. In an embodiment wherein the player-accessible value input device 118 comprises a biometric player information reader, transactions such as an input of value to the handheld device, a transfer of value from one player account or source to an account associated with the handheld gaming machine 110, or the execution of another transaction, for example, could all be authorized by a biometric reading, which could comprise a plurality of biometric readings, from the biometric device.

Alternatively, to enhance security, a transaction may be optionally enabled only by a two-step process in which a secondary source confirms the identity indicated by a primary source. For example, a player-accessible value input device 118 comprising a biometric player information reader may require a confirmatory entry from another biometric player information reader 152, or from another source, such as a credit card, debit card, player ID card, fob key, PIN number, password, hotel room key, etc. Thus, a transaction may be enabled by, for example, a combination of the personal identification input (e.g., biometric input) with a secret PIN number, or a combination of a biometric input with a fob input, or a combination of a fob input with a PIN number, or a combination of a credit card input with a biometric input. Essentially, any two independent sources of identity, one of which is secure or personal to the player (e.g., biometric readings, PIN number, password, etc.) could be utilized to provide enhanced security prior to the electronic transfer of any funds. In another aspect, the value input device 118 may be provided remotely from the handheld gaming machine 110.

The player input device 124 comprises a plurality of push buttons on a button panel for operating the handheld gaming machine 110. In addition, or alternatively, the player input device 124 may comprise a touch screen mounted to a primary display 114 and/or secondary display 116. In one aspect, the touch screen is matched to a display screen having one or more selectable touch keys 130 selectable by a user's touching of the associated area of the screen using a finger or a tool, such as a stylus pointer. A player enables a desired function either by touching the touch screen at an appropriate touch key 130 or by pressing an appropriate push button 126 on the button panel. The touch keys 130 may be used to implement the same functions as push buttons 126. Alternatively, the push buttons may provide inputs for one aspect of the operating the game, while the touch keys 130 may allow for input needed for another aspect of the game. The various components of the handheld gaming machine 110 may be connected directly to, or contained within, the casing 112, as seen in FIG. 1b, or may be located outboard of the casing 112 and connected to the casing 112 via a variety of hardwired (tethered) or wireless connection methods. Thus, the handheld gaming machine 110 may comprise a single unit or a plurality of interconnected parts (e.g., wireless connections) which may be arranged to suit a player's preferences.

The operation of the basic wagering game on the handheld gaming machine 110 is displayed to the player on the primary display 114. The primary display 114 can also display the bonus game associated with the basic wagering game. The primary display 114 preferably takes the form of a high resolution LCD, a plasma display, an LED, or any other type of display suitable for use in the handheld gaming machine 110. The size of the primary display 114 may vary from, for example, about a 2-3″ display to a 15″ or 17″ display. In at least some aspects, the primary display 114 is a 7″-10″ display. As the weight of and/or power requirements of such displays decreases with improvements in technology, it is envisaged that the size of the primary display may be increased. Optionally, coatings or removable films or sheets may be applied to the display to provide desired characteristics (e.g., anti-scratch, anti-glare, bacterially-resistant and anti-microbial films, etc.). In at least some embodiments, the primary display 114 and/or secondary display 116 may have a 16:9 aspect ratio or other aspect ratio (e.g., 4:3). The primary display 114 and/or secondary display 116 may also each have different resolutions, different color schemes, and different aspect ratios.

As with the free standing gaming machine 10, a player begins play of the basic wagering game on the handheld gaming machine 110 by making a wager (e.g., via the value input device 18 or an assignment of credits stored on the handheld gaming machine via the touch screen keys 130, player input device 124, or buttons 126) on the handheld gaming machine 110. In at least some aspects, the basic game may comprise a plurality of symbols arranged in an array, and includes at least one payline 132 that indicates one or more outcomes of the basic game. Such outcomes are randomly selected in response to the wagering input by the player. At least one of the plurality of randomly selected outcomes may be a start-bonus outcome, which can include any variations of symbols or symbol combinations triggering a bonus game.

In some embodiments, the player-accessible value input device 118 of the handheld gaming machine 110 may double as a player information reader 152 that allows for identification of a player by reading a card with information indicating the player's identity (e.g., reading a player's credit card, player ID card, smart card, etc.). The player information reader 152 may alternatively or also comprise a bar code scanner, RFID transceiver or computer readable storage medium interface. In one presently preferred aspect, the player information reader 152, shown by way of example in FIG. 1b, comprises a biometric sensing device.

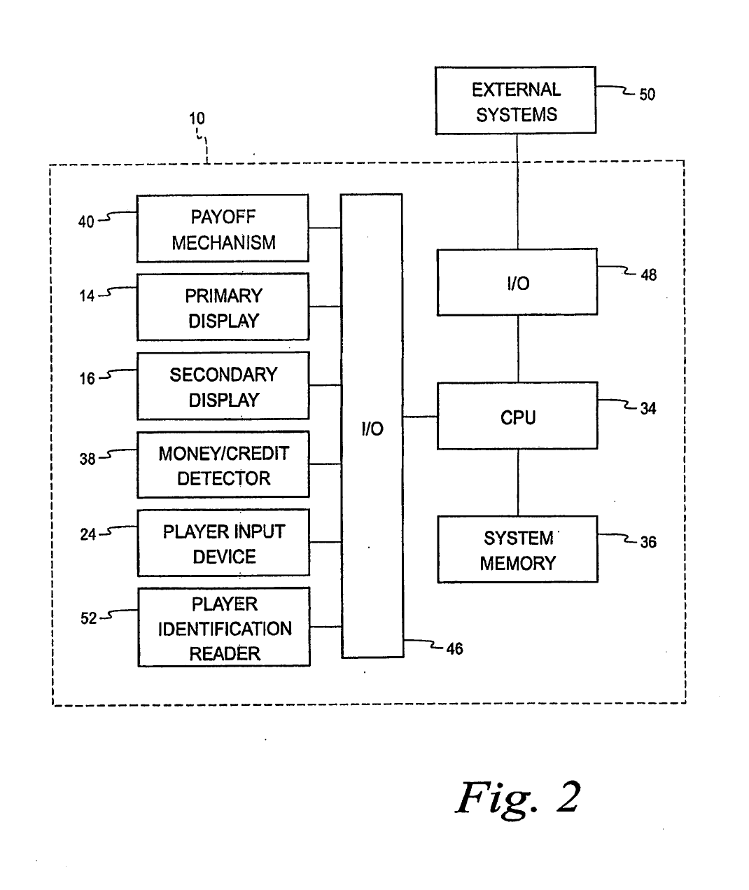

Turning now to FIG. 2, the various components of the gaming machine 10 are controlled by a central processing unit (CPU) 34, also referred to herein as a controller or processor (such as a microcontroller or microprocessor). To provide gaming functions, the controller 34 executes one or more game programs stored in a computer readable storage medium, in the form of memory 36. The controller 34 performs the random selection (using a random number generator (RNG)) of an outcome from the plurality of possible outcomes of the wagering game. Alternatively, the random event may be determined at a remote controller. The remote controller may use either an RNG or pooling scheme for its central determination of a game outcome. It should be appreciated that the controller 34 may include one or more microprocessors, including but not limited to a master processor, a slave processor, and a secondary or parallel processor.

The controller 34 is also coupled to the system memory 36 and a money/credit detector 38. The system memory 36 may comprise a volatile memory (e.g., a random-access memory (RAM)) and a non-volatile memory (e.g., an EEPROM). The system memory 36 may include multiple RAM and multiple program memories. The money/credit detector 38 signals the processor that money and/or credits have been input via the value input device 18. Preferably, these components are located within the housing 12 of the gaming machine 10. However, as explained above, these components may be located outboard of the housing 12 and connected to the remainder of the components of the gaming machine 10 via a variety of different wired or wireless connection methods.

As seen in FIG. 2, the controller 34 is also connected to, and controls, the primary display 14, the player input device 24, and a payoff mechanism 40. The payoff mechanism 40 is operable in response to instructions from the controller 34 to award a payoff to the player in response to certain winning outcomes that might occur in the basic game or the bonus game(s). The payoff may be provided in the form of points, bills, tickets, coupons, cards, etc. For example, in FIG. 1a, the payoff mechanism 40 includes both a ticket printer 42 and a coin outlet 44. However, any of a variety of payoff mechanisms 40 well known in the art may be implemented, including cards, coins, tickets, smartcards, cash, etc. The payoff amounts distributed by the payoff mechanism 40 are determined by one or more pay tables stored in the system memory 36.

Communications between the controller 34 and both the peripheral components of the gaming machine 10 and external systems 50 occur through input/output (I/O) circuits 46, 48. More specifically, the controller 34 controls and receives inputs from the peripheral components of the gaming machine 10 through the input/output circuits 46. Further, the controller 34 communicates with the external systems 50 via the I/O circuits 48 and a communication path (e.g., serial, parallel, IR, RC, 10bT, etc.). The external systems 50 may include a gaming network, other gaming machines, a gaming server, communications hardware, or a variety of other interfaced systems or components. Although the I/O circuits 46, 48 may be shown as a single block, it should be appreciated that each of the I/O circuits 46, 48 may include a number of different types of I/O circuits.

Controller 34, as used herein, comprises any combination of hardware, software, and/or firmware that may be disposed or resident inside and/or outside of the gaming machine 10 that may communicate with and/or control the transfer of data between the gaming machine 10 and a bus, another computer, processor, or device and/or a service and/or a network. The controller 34 may comprise one or more controllers or processors. In FIG. 2, the controller 34 in the gaming machine 10 is depicted as comprising a CPU, but the controller 34 may alternatively comprise a CPU in combination with other components, such as the I/O circuits 46, 48 and the system memory 36. The controller 34 may reside partially or entirely inside or outside of the machine 10. The control system for a handheld gaming machine 110 may be similar to the control system for the free standing gaming machine 10 except that the functionality of the respective on-board controllers may vary.

The gaming machines 10,110 may communicate with external systems 50 (in a wired or wireless manner) such that each machine operates as a “thin client,” having relatively less functionality, a “thick client,” having relatively more functionality, or through any range of functionality therebetween. As a generally “thin client,” the gaming machine may operate primarily as a display device to display the results of gaming outcomes processed externally, for example, on a server as part of the external systems 50. In this “thin client” configuration, the server executes game code and determines game outcomes (e.g., with a random number generator), while the controller 34 on board the gaming machine processes display information to be displayed on the display(s) of the machine. In an alternative “thick client” configuration, the server determines game outcomes, while the controller 34 on board the gaming machine executes game code and processes display information to be displayed on the display(s) of the machines. In yet another alternative “thick client” configuration, the controller 34 on board the gaming machine 110 executes game code, determines game outcomes, and processes display information to be displayed on the display(s) of the machine. Numerous alternative configurations are possible such that the aforementioned and other functions may be performed onboard or external to the gaming machine as may be necessary for particular applications. It should be understood that the gaming machines 10,110 may take on a wide variety of forms such as a free standing machine, a portable or handheld device primarily used for gaming, a mobile telecommunications device such as a mobile telephone or personal daily assistant (PDA), a counter top or bar top gaming machine, or other personal electronic device such as a portable television, MP3 player, entertainment device, etc.

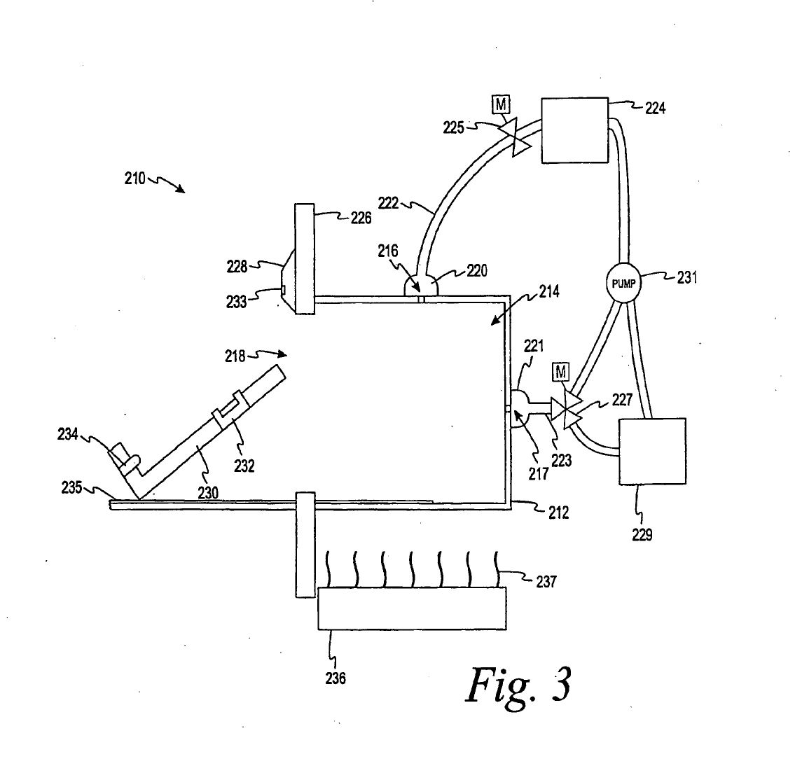

Referring now to FIG. 3, a handheld gaming machine sanitizing system (sanitizing system) 210 is illustrated. The sanitizing system 210 is adapted to sanitize the handheld gaming machine (HHGM) 110. The sanitizing system 210 includes a housing 212 defining a chamber 214, a gas inlet aperture 216, an optional gas exit aperture 217, and an opening 218. Although the system 210 will be referred to as a sanitizing system 210, it is understood that the system 210 in all of its embodiments in accordance with the present invention can provide sanitizing, disinfecting, and/or sterilizing effects on HHGMs. Therefore, unless specifically expressed otherwise, the terms sanitize, santized, and sanitizing, as used herein, shall be understood to generally include sanitizing to varying degrees, disinfecting to varying degrees, and sterilizing effects on HHGMs in accordance with particular applications of the present invention.

The chamber 214 of the housing 212 provides the specific environment wherein the HHGM 110 is sanitized. The gas inlet aperture 216 is associated with a fitting 220 that connects the chamber 214 to a gas supply line 222. The gas supply line 222 is, in turn, associated with a gas supply 224 comprising a sanitizing gas. A flow control device 225 is disposed in the gas supply line 222 between the housing 212 and the gas supply 224 to regulate the flow of the sanitizing gas from the gas supply 224 to the chamber 214. Optionally, the flow control device 225 is integrated with the fitting 220. A plurality of flow control devices 225 may also be provided. A control system (not shown) is provided to control the flow control device(s) 225. The flow control device 225 may include, for example, but are not limited to, a manually-operated valve, a motor-operated valve, a solenoid-operated valve, a check valve, or any other actuatable valve.

The optional gas exit aperture 217 is associated with a fitting 221 that connects the chamber 214 to a gas exit line 223. The gas exit line 223 leads to a flow control device 227. The flow control device 227, optionally in combination with an associated control system, is actuated following completion of the sanitizing process to discharge the gases present in the chamber 214. Specifically, the flow control device 227 permits discharge to a lower pressure source, such as a gas recycling unit 229 or a pump inlet 231. In at least some aspects, the discharge of the atmosphere of the chamber 214 following the sanitizing process may occur by opening a door 226 to vent directly to the surroundings.

The opening 218 in the housing 212 affords the unobstructed entrance and exit of the HHGM 110 to and from the chamber 214. The door 226 is provided to selectively seal the opening 218. When the door 226 is open, the HHGM 110 may enter and exit the chamber 214 of the housing 212. When closed, the door 226 inhibits and/or prevents the leakage of gas from the chamber 214. In at least some aspects, the door 226 includes a display 228 configured to display various types of information to a user of the sanitizing system 210. The door 226 may also or alternatively include an input device 233, such as a key pad or a key board to input information to the sanitizing system 210. The display panel 228 or the input device 233 may alternatively be provided elsewhere on or adjacent to the housing 212, or even remote from the sanitizing system 210.

A stand 230 is provided to accommodate the HHGM 110 in the sanitizing system 210. In a presently preferred aspect, the stand 230 is movable from a position outside the chamber 214 to a position inside the chamber 214 and includes a locking device 232 and a charging bay 234. Accordingly, the sanitizing system 210 also includes rails 235 to assist the movement of the stand 230. The sanitizing system 210 may alternatively include a slidable plate on bearings or other mechanisms to move the stand 230 between a position outside the chamber 214 and a position inside the chamber 214. The locking device 232 enables a secure connection between the stand 230 and the HHGM 110. The charging bay 234 is provided to charge the battery of the HHGM 110 while the HHGM 110 is being sanitized. In accord with some aspects, the HHGM 110 has components that correspond to the locking device 232 and the charging bay 234 of the sanitizing system. In accord with other aspects, the locking device 232 may be configured to adapt to HHGDs 110 of varying geometry and configurations.

In some embodiments, as illustrated in FIG. 3, ultraviolet lamps 236 are disposed adjacent to or within the chamber 214 to direct ultraviolet light 237 into the chamber 214 to provide a sanitizing effect.

During game play, through handling and other environmental contact, the HHGM 110 may be in an unacceptable state of cleanliness. For example, the HHGM 110 may be brought into a washroom, food and/or drink may be spilled on the HHGM 110, the HHGM 110 may come into contact with a substantial amount of smoke or other foul odors, a user may have unclean hands when handling the HHGM 110, or a sick person may handle or otherwise contaminate the HHGM 110. The sanitizing system 210 is configured to sanitize, disinfect, and/or sterilize the HHGM 110.

The HHGM 110 may advantageously be pre-treated before use of the sanitizing system 210. For example, if a liquid were spilled on the HHGM 110, an attendant may wipe or brush the HHGM 110 before putting the HHGM 110 into the sanitizing system 210. A cleaning solution may optionally be provided in combination therewith.

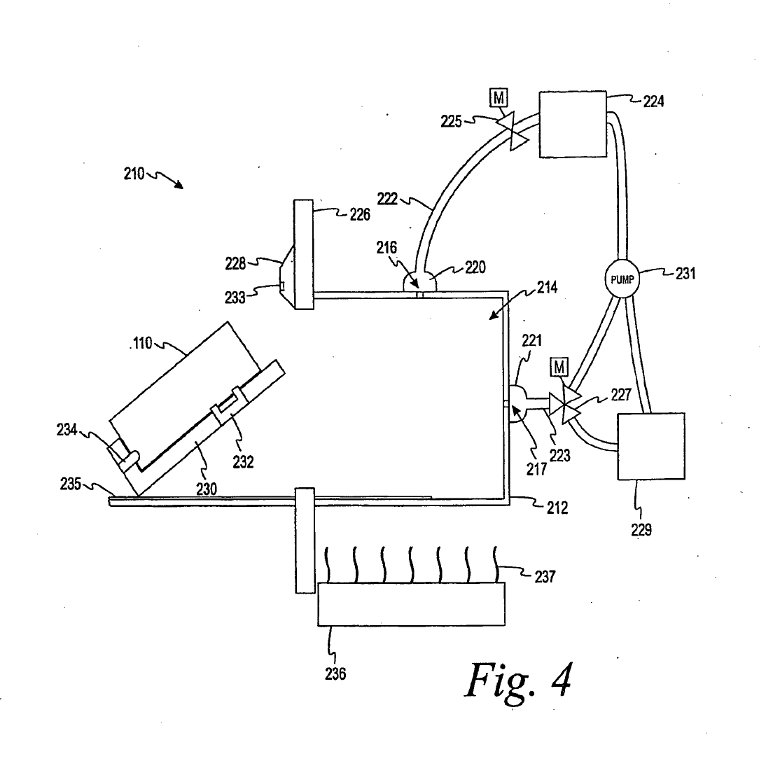

In at least some aspects, following such optional pre-treatment, the HHGM 110 is placed in the chamber 214. More particularly, in accord with at least some aspects, the HHGM 110 is placed in the stand 230, as illustrated in FIG. 4. Once in the stand 230, the HHGM 110 may be optionally locked into place via the locking device 232. Furthermore, the charging bay 234 of the stand 230 will advantageously re-charge the battery of the HHGM 110 upon activation of the charging bay 234, via an input device 233, by the user. However, in some embodiments, the charging bay 234 re-charges the battery of the HHGM 110 automatically upon insertion of the HHGM 110 into the stand 230.

Whether the HHGM 110 is locked or unlocked or whether the HHGM 110 is being charged or not may be displayed to the user by the display 228, which comprises a touch screen display in at least some aspects. The display 228 is preferably configured to display status information, such as, but not limited to, whether the sanitizing system 210 is ready for sanitizing, whether the sanitizing system 210 is in the process of sanitizing, the status of the sanitization process, and/or whether the chamber 214 is empty. Other types of status information or instructions may also be displayed by the display 228, an indicator, and/or input device 233.

Furthermore, prior to activation of the sanitizing system 210, or during the sanitizing process, user preferences may be entered. These user preferences may include, but are not limited to, whether or not to charge the battery of the HHGM 110 during the sanitization process, the duration of the sanitization process, and/or degree of sanitization desired may be input into the input device 233.

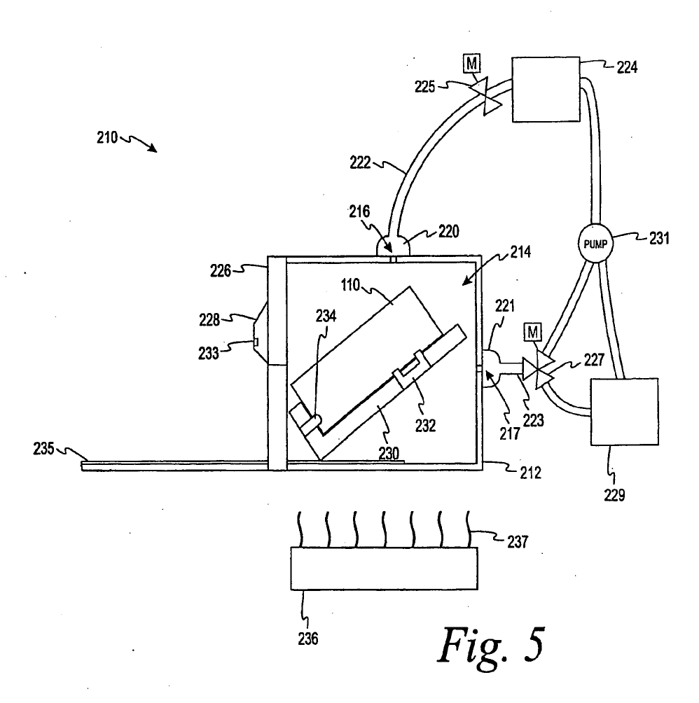

Now, referring to FIG. 5, the stand 230 and the HHGM 110 are shown to be inserted into the chamber 214 of the housing 212. In this operational configuration, the door 226 is closed, manually or automatically, to substantially seal the chamber 214. The sanitizing system 210 is then enabled for automatic operation or for operation subsequent to a user input via the input device 233. In one embodiment, the air initially within the chamber 214 may be partially or substantially evacuated via the flow control device 227 and the gas exit line 223 to prepare for the introduction of the sanitizing gas from the gas supply 224 into the chamber 214.

Upon activation (or deactivation, if appropriate), the flow control device 225 allows the sanitizing gas from the gas supply 224 to enter the chamber 214. In some embodiments, the flow control device 225 is automatically activated when the door 226 closes to allow the sanitizing gas to enter the chamber 214. The flow control device 225 is preferably controlled to permit a predetermined amount of sanitizing gas into the chamber 214. The flow control device 227 is preferably controlled to permit the sanitizing gas to reside in the chamber 214 for a pre-determined length of time. The length of time may be automatically selected in accord, for example, to one of a plurality of programmed sequences, or user-selectable. The amount of sanitizing gas that enters the chamber 214, in combination with the selected duration of the process, should be sufficient to sanitize the HHGM 110 to a desired degree.

By “submersing” the HHGM 110 in a sanitizing gas, the gas is able to penetrate areas that wet cleaning processes may not practicably reach and saturate the surfaces thereof (e.g., both inside and outside of the HHGM 110) to sanitize the HHGM 110 to a desired degree.

The term sanitizing gas, as used herein, includes any type of “gas” that has a sanitizing, disinfecting, or sterilizing effect. The sanitizing gas, in at least some aspects, is selected to provide at least a reduction in microbial count as part of a sanitizing application. Therefore, the sanitizing gas provides, in such aspects, anti-microbial properties to reduce microbial counts, including, but not limited to, reduction in bacteria, fungi, yeast, mold, and mildew, counts. In some embodiments, it may be desirable that the sanitizing gas provide anti-viral and anti-pathogen properties to attack contaminants comprising viruses and other pathogens. Therefore, the sanitizing gas may additionally provide anti-viral properties to attack these viruses and/or other pathogens. Accordingly, the term sanitizing gas is used herein to comprise properties that can sanitize and disinfect (i.e., reduce microbial and viral counts), and/or sterilize (i.e., substantially, if not entirely, eliminate counts thereof), with respect to the HHGM 110 being sanitized by the sanitizing gas.

In one embodiment, the sanitizing gas includes ozone gas or a mixture of ozone gas with one or more other gases. Ozone, in varying concentrations, as a treatment for the HHGM 110, can provide beneficial sanitizing, disinfecting, and sterilizing, effects thereto. Other gases having biocidal (e.g., bactericidal, fungicidal, etc.) properties that may be used for disinfection and sterilization include, but are not limited to, chlorine dioxide, ethylene oxide, hydrogen peroxide vapor, and/or hydrogen peroxide plasma. Other sanitizing gases may also be used.

In other embodiments, the sanitizing gas may comprise a “dry” steam vapor comprising a high temperature (approximately at least 200° F.), low moisture vapor. In some aspects, the vapor comprises a low percentage of water (preferably about 5-6%, but other percentages may be used).

As illustrated and mentioned above, one or more ultraviolet lamps 236 are disposed adjacent to or within the chamber 214 to direct ultraviolet light 237 into the chamber 214. Ultraviolet radiation (e.g., in the 185-300 nm range) is known to be effective in killing microorganisms such as airborne and surface bacteria, viruses, yeasts, and molds. A sanitizing gas is not necessary for use in combination with the ultraviolet lamps 236. However, a sanitizing gas, such as ozone gas, provided in combination with an ultraviolet lamp 236 is believed to be particularly effectuous.

Following sanitization of the HHGM 110 by the sanitizing gas and/or ultraviolet lamps 236, the flow control device 227 is actuated to evacuate the remaining sanitizing gas from the chamber 214 via the gas exit aperture 217 and the gas exit line 223. The output gas is then routed via the flow control device 227, or other flow control device, to a predetermined destination, such as a pump inlet 231 for recirculation back to the gas supply 224 or to a gas recycling unit 229.

Once the sanitizing gas is evacuated from the chamber 214, the user is preferably notified that sanitization is complete via the display 228, indicator light, buzzer, or automatic opening of the door 226. The user may then open the door 226, if not automatically opened, and remove the sanitized HHGM 110.

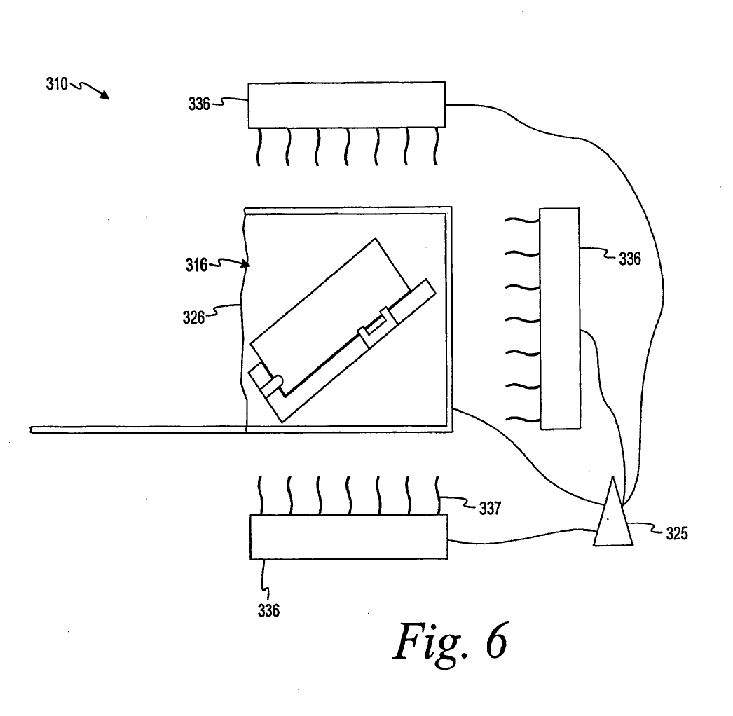

A sanitizing system not utilizing a sanitizing gas is also contemplated in the present invention. Referring now to FIG. 6, a sanitizing system 310 utilizing only ultraviolet radiation 337 via ultraviolet lamps 336 is illustrated. In this embodiment, a door is not necessary and any type of partition 326 may be provided that at least substantially occludes opening 316. A control system 325 is preferably provided to activate/deactivate the ultraviolet lamps 336, via user-input or automatically. In another embodiment, the ultraviolet lamps 336 are manually actuated. The ultraviolet lamps 336 produce a sanitizing effect because, as mentioned above, ultraviolet light, particularly in the germicidal wavelength 185-254 nm kills microorganisms. Furthermore, germicidal ultraviolet lamps that generate energy between about 185 nm and 254 nm is believed to produce an abundant amount of ozone in the air.

In some other embodiments, the sanitizing system 210, 310 does not include a door 226 or partition 326, but remains generally open. The opposite end of the door 226 and/or partition 326 of the sanitizing system 210, 310 may also remain open with a moving conveyor belt running therethrough.

Each of these embodiments and obvious variations thereof is contemplated as falling within the spirit and scope of the claimed invention, which is set forth in the following claims.

What is claimed is:

1. A sanitizing system for a handheld gaming device, comprising:
a housing defining at least a chamber, a gas inlet aperture and an opening for receiving a handheld gaming device;
a door configured to seal the opening;
a gas supply comprising a sanitizing gas connected to the gas inlet aperture; and
a control system configured to selectively introduce gas from the gas supply into the chamber.

2. The sanitizing system of claim 1, wherein the control system is adapted to maintain the gas within the chamber for a predetermined period of time

3. The sanitizing system of claim 2, wherein the predetermined period of time is sufficient to substantially sanitize the handheld gaming device.

4. The sanitizing system of claim 1, further comprising a stand for the handheld gaming device disposed in the chamber.

5. The sanitizing system of claim 4, wherein the stand comprises a locking device for securing the handheld gaming device to the stand.

6. The sanitizing system of claim 4, wherein the stand is movable from a first position to a second position, the first position being outside the housing, the second position being inside the chamber.

7. The sanitizing system of claim 6, wherein the stand includes a charging bay to re-charge the handheld gaming device.

8. The sanitizing system of claim 1, wherein the gas comprises ozone.

9. The sanitizing system of claim 1, further comprising at least one ultraviolet lamp disposed at least one of adjacent and within the chamber to direct ultraviolet light into the chamber.

10. The sanitizing system of claim 1, wherein the housing further defines a gas outlet aperture, wherein the control system further being configured to selectively remove gas from the gas supply into the chamber.

11. The sanitizing system of claim 10, wherein the gas is removed to at least one of a gas recycling unit and the gas supply

12. The sanitizing system of claim 1, further comprising at least one of a status display panel, a touchscreen, status indicator lights, a key pad, and a key board.

13. A method for sanitizing a handheld gaming device, comprising:
moving the handheld gaming device into a chamber;
substantially sealing the chamber; and
sanitizing the handheld gaming device.

14. The method of claim 13, further comprising the act of introducing a sanitizing gas into the substantially sealed chamber.

15. The method of claim 14, wherein the gas comprises ozone.

16. The method of claim 14, further comprising the act of removing the gas from the substantially sealed chamber.

17. The method of claim 13, further comprising the act of introducing ultraviolet radiation into the substantially sealed chamber.

18. The method of claim 17, wherein the ultraviolet radiation is in the range of 200-300 nanometers.

19. The method of claim 14, further comprising the act of introducing ultraviolet radiation into the substantially sealed chamber.

20. The method of claim 13, further comprising the act of charging a battery in the handheld gaming device during the act of sanitizing.

21. A sanitizing system for a handheld gaming device, comprising:
a housing defining at least a chamber and an opening for receiving a handheld gaming device;
a partition configured to occlude the opening; and
at least one ultraviolet lamp disposed at least one of adjacent and within the chamber to introduce ultraviolet light into the chamber to at least partially sanitize the handheld gaming device disposed therein.

22. The sanitizing system of claim 21, further comprising a control system configured to selectively activate and deactivate the at least one ultraviolet lamps.

23. A method for sanitizing a handheld gaming device, comprising:
placing the handheld gaming device in a controlled environment; and
sanitizing the handheld gaming device in the controlled environment.

24. The method of claim 23, wherein the controlled environment includes at least one of radiation, radiation and gas, and gas.

25. The method of claim 23, wherein the controlled environment is sealed after said placing.
